(19) United States
(12) Patent Application Publication
(10) Pub. No.: US 2002/0006447 A1
(43) Pub. Date: Jan. 17, 2002

(54) Anti-chlamydia agent

(75) Inventors: Tsutomu Yamazaki, Tokyo (JP); Yukihiko Hara, Fujieda-shi (JP)

Correspondence Address: FRISHAUF, HOLTZ, GOODMAN,, LANGER & CHICK, P.C., 25th Floor, 767 Third Avenue, New York, NY, 10017-2023, US

(73) Assignee: Mitsui Norin Co., Ltd., Tokyo (JP)

(21) Appl. No.: 09/910,499

(22) Filed: Jul. 19, 2001

Related U.S. Application Data
(63) Continuation of application No. 09/424,725, filed on Nov. 30, 1999.

(30) Foreign Application Priority Data
Apr. 21, 1998 (JP) 125392/1998

(51) Int. Cl. A61K 35/78; A61K 31/7048; A61K 31/353

(52) U.S. Cl. 424/729; 514/27; 424/456

(57) ABSTRACT

The present invention relates to an anti-Chlamydia agent characterized by containing tea polyphenol and to a method for preventing or treating a Chlamydia infectious disease, characterized by comprising administering a composition containing tea polyphenol in an amount effective for the therapy of a Chlamydia infectious disease on an affected part of a patient.

The tea polyphenol is a component of tea, natural substance. Tea has been used widely since old time as a beverage so that there is no problem on safety. Therefore, tea polyphenol has no harmful side effects on human body and no risk of emergence of resistant strains.

### TECHNICAL FIELD

The present invention relates to an anti-Chlamydia agent characterized by containing tea polyphenol.

### BACKGROUND ART

Bacteria belonging to genus Chlamydia are of a spherical or ellipsoidal shape of 0.2 to 1.5 μm and are unique bacteria which are obligate parasites in cells of eucaryotes.

Infectious body, which is an ecotype outside host cells, enters into the vacuoles of host cells and converted therein into particles called reticular structural body due to phagocytosis. The reticular formation increases in number by division and matures into an infectious body in a later stage of infection.

Chlamydia are known as pathogens for trachoma and inclusion conjunctivitis (Chlamydia trachomatis), for parrot disease (C. psittaci), as well as known as pathogens for lung fever (pneumonia), sore throat (pharyngitis), bronchitis, sinusitis, otitis media (C. pneumoniae) or others in child. C. trachomatis is a major pathogenic microorganism for sexually transmitted diseases prevailing worldwide.

For the therapy of Chlamydia infectious diseases, in particular C. trachomatis infectious diseases, oral administration of antibiotics is usually used. In this case, the antibiotics which can be used include doxycycline and mynocycline of tetracycline group, clarithromycin of macrolide group, ofloxacin, tosufloxacin, and sparfloxacin of neuquinolone group, etc. For the therapy, oral administration is usually continued for about 2 weeks. However, in the case of therapy with antibiotics, the problem of side effects is associated. Hence, drugs of tetracycline group and new quinolone group cannot be administered to pregnant women. Moreover, administration of antibiotics is always accompanied by the risk of the emergence of drug-resistant strains.

Accordingly, an object of the present invention is to find an anti-Chlamydia agent which has no side effect nor the fear of emergence of resistant strains out of natural substances and to provide it.

The inventors of the present invention have made intensive investigation in order to achieve the above object and as a result they have found that tea polyphenol contained in tea leaves has a remarkable proliferation inhibiting activity against bacteria belonging to genus Chlamydia, thus completing the present invention based on this finding.

Tea used in the present invention has been used as beverage from old times and has no problem on its safety.

### DISCLOSURE OF THE INVENTION

The present invention provides an anti-Chlamydia agent containing tea polyphenol.

Further, the present invention provides a method for treating a Chlamydia infectious disease comprising administering a composition containing tea polyphenol in an amount effective for the therapy of a Chlamydia infectious disease on an affected part of a patient.

### BEST MODE FOR CARRYING OUT THE INVENTION

In the present invention, tea means leaf, stem, xylem, root, and seed obtained from tea tree (Camellia sinensis) or mixtures of these. Usually, tea leaves for beverages are generally used as a raw material.

Tea leaves for beverages include various kinds depending on the method for their production, for example, fermented tea such as black tea and pooar tea, semi-fermented tea such as oolong tea and paochong tea, and, non-fermented tea such as green tea, and mixtures of these. In the present invention, any of them may be used.

Tea polyphenol which can be used in the present invention include tea itself containing said tea polyphenol, extracts from the above tea with water, hot water, organic solvents, hydrous organic solvents, etc., or mixtures thereof. Further, there can be used high tea polyphenol content preparations obtained by purifying tea extracts to a desired degree by organic solvent fractionation or chromatography using adsorbing resins. These methods are described in Japanese Patent Publication Nos. Hei 1-44234, Hei 2-12474, and Hei 2-22755, Japanese Patent Kokai Nos. Hei 4-20589, Hei 5-260907, and Hei 8-109178, etc.

Tea polyphenol contained in the tea extracts and high tea polyphenol content preparations thus obtained specifically includes catechins, that is, (+)-catechin, (−)-catechin, (+)-gallocatechin, (+)-epigallocatechin, (+)-gallocatechin gallate, (+)-epigallocatechin gallate, (−)-epicatechin, (−)-epicatechin gallate, (−)-catechin gallate, (−)-epigallocatechin, (−)-gallocatechin, (−)-epigallocatechin gallate, (−)-gallocatechin gallate, etc., and teaflavins, that is, teaflavin monogallate A, teaflavin monogallate B, teaflavin digallate, free teaflavin, etc. They are used singly or in combination.

The above tea polyphenol may be commercially available products, for example, those which contain catechins as a major ingredient, such as trade name: Polyphenon 60 (manufactured by Mitsui Norin Co., Ltd., tea polyphenol content: 60% or more), trade name: Polyphenon 30 (manufactured by Mitsui Norin Co., Ltd., tea polyphenol content: 30% or more), trade name: Polyphenon 70S (manufactured by Mitsui Norin Co., Ltd., tea polyphenol content: 70% or more), trade name: Polyphenon E (manufactured by Mitsui Norin Co., Ltd., tea polyphenol content: 80% or more), etc. Those which contain teaflavins as a major ingredient include trade name: Polyphenon TF (manufactured by Mitsui Norin Co., Ltd., composition: 16.8% teaflavin, 19.5% teaflavin monogallate A, 16.1% teaflavin monogallate B, 31.4% teaflavin digallate), etc.

The anti-Chlamydia agent of the present invention can be applied to bacteria belonging to genus Chlamydia such as C. pneumoniae, C. psittaci, and C. pecorum, as well as to C. trachomatis.

In the present invention, upon using the above tea polyphenol, it is combined with a suitable solubilizing agent, suspending agent, base material, or the like and used as a composition in the form of cream, paste, gel, milky lotion, liquid, etc. For example, tea polyphenol and/or tea polyphenol-containing material may be dissolved and/or suspended in purified water, physiological saline, hydrous ethanol etc., and sprayed and/or coated on the affected part such as mucous membrane of respiratory tract, etc. For trachoma, inclusion conjunctivitis, etc., tea polyphenol may be dissolved in purified water, buffer solution or the like and used as eyedroppers or may be mixed with a base material for ointment and used as eyepaste. Also, it is possible to mix tea polyphenol with cream, paste, gel, ointment, etc. and to coat the affected part such as epithelium of cervical canal, etc. with it.

The bases for cream, paste, gel, and ointment in the present invention include, for example, hydrocarbons such as white vaseline, yellow vaseline, paraffin, liquid paraffin, squalane, and ceresine; higher fatty acids such as lauric acid, stearic acid, myristic acid, palmitic acid, oleic acid, and linolic acid; higher fatty acid alcohol such as stearyl alcohol, oleyl alcohol, lauryl alcohol, cetyl alcohol, and lanolin alcohol; fatty acid esters such as sorbitan sesquioleate, isopropyl myristate, isopropyl palmitate, and glycerin monostearate; waxes such as beeswax, white beeswax, and lanolin; oils and fats such as avogado oil, olive oil, cacao oil, sesame oil, soy bean oil, castor oil, macadamia nut oil, mink oil, yolk oil, beef tallow, and lard; high molecular compounds such as gum arabi, tragacanth gum, guar gum, karaya gum, dextrin, gelatin, carrageenan, shellac, rosin, casein, sodium carboxymethylcellulose, methylcellulose, ethylcellulose, sodium alginate, nitrocellulose, polyvinyl alcohol, polyvinylpyrrolidone, sodium polyacrylate, polyvinyl methyl ether, lauromacrogol, polyamide resins, and silicone oil, and one or more of these may be selected appropriately and used.

To the above preparations can be added are humectants such as glycerin, propylene glycol, 1,3-butylene glycol, polyethylene glycol, sodium dl-pyrrolidonecarboxylate, sodium lactate, sorbitol, sodium hyaluronate; inorganic substances such as bentonite, kaolin, zinc oxide, and titanium oxide; stabilizers such as methyl paraoxybenzoate, ethyl paraoxybenzoate, propyl paraoxybenzoate, and butyl paraoxybenzoate; antiseptics such as benzalkonium chloride, benzethonium chloride, citric acid, sodium citrate, paraoxybenzoic acid, and boric acid; surfactants such as polyoxyethylene-hydrated castor oil, and known percutaneous absorption promoter, etc., if desired.

In these cases, the concentration of tea polyphenol in the drug may vary depending on the symptom and age of patients, site of use, method of use, etc. and is not limited particularly. Usually, when used in the form of liquid, milky lotion, etc., 0.2 to 50 mg/ml, preferably 1.6 to 10 mg/ml. When used in the form of cream, paste, gel, ointment, etc., the concentration of tea polyphenol in the drug is 0.2 to 200 mg/g, preferably 10 to 100 mg/g.

When the anti-Chlamydia agent of the present invention is used, it is desirable to continue the therapy for a certain period of time, e.g., 2 to 4 weeks, taking into consideration the unique growth cycle of Chlamydia and prevention of recurrence. Although the frequency of use of the agent during that time may vary depending on the factors, such as symptom of the patient, site of use, method of use, concentration of tea polyphenol used, etc., it is possible to continue daily use of 1 to 10 times a day.

The tea polyphenol which is an active ingredient of the anti-Chlamydia agent of the present invention is highly safe so that it can be used for preventive purposes.

Hereafter, the present invention will be described more specifically by examples. However, the present invention is not limited thereto.

### EXAMPLE 1

As test Chlamydia was used C. trachomatis strain. Chlamydia capable of forming 10⁴ inclusion bodies was incubated at 37° C. for 30 minutes, 60 minutes or 90 minutes in a SPG (sucrose phosphate glutamate) solution containing tea polyphenol of various concentrations (trade name: Polyphenon 70S manufactured by Mitsui Norin Co., Ltd., catechin content: (−)-epigallocatechin 18.3%, (−)-epicatechin 8.6%, (−)-epigallocatechin gallate 35.9%, (−)-epicatechin gallate 11.2%, (−)-gallocatechin gallate 3.5%). As a control, Chlamydia was incubated similarly in SPG solution containing no tea polyphenol.

After completion of the incubation, each solution was inoculated onto HeLa 229 cell cultivated by monolayer culture and adsorbed by centrifugation at 1,500 rpm for 60 minutes. Thereafter, the inoculum was removed and culture medium for Chlamydia (Eagle Minimum Essential Culture Medium containing 1 μg/ml cycloheximide) was added and cultivation was performed at 37° C. for 72 hours. After completion of cultivation, the culture medium was removed and the cells were fixed with methanol and then stained with fluorescein isothiocyanate (FITC)-labeled anti-C. trachomatis monoclonal antibody, followed by counting the number of inclusion bodies of C. trachomatis.

The results obtained are shown in Table 1. In the table, the number of inclusion bodies in each treatment is indicated as a relative value with respect to the number of inclusion bodies in the control. As will be apparent from the table, in each group the number of C. trachomatis inclusion bodies decreased except when ed for 30 minutes after addition of 0.2 mg/ml of tea polyphenol. In particular, when incubated for 90 minutes, addition of 1.6 mg/ml or more of tea polyphenol completely inhibited the growth of C. trachomatis.

TABLE 1

| Incubation | Concentration of Tea Polyphenol (mg/ml) | | | | | | |
|---|---|---|---|---|---|---|---|
| time (minute) | 0 | 0.2 | 0.4 | 0.8 | 1.6 | 3.2 | 6.4 |
| 30 | 1.00 | 1.00 | 0.58 | 0.34 | 0.18 | 0.10 | 0.05 |
| 60 | 1.00 | 0.87 | 0.41 | 0.18 | 0.06 | 0.02 | 0.02 |
| 90 | 1.00 | 0.55 | 0.07 | 0.03 | 0 | 0 | 0 |

### EXAMPLE 2

As test Chlamydia was used C. trachomatis strain. Chlamydia capable of forming 10⁴ inclusion bodies was incubated at 37° C. for 90 minutes in a SPG solution containing (−)-epicatechin gallate or (−)-epigallocatechin gallate in various concentratIons.

After completion of the incubation, each solution was inoculated onto Hela 229 cell cultivated by monolayer culture and adsorbed by centrifugation at 1,500 rpm for 60 minutes. Thereafter, the inoculum was removed and culture medium for Chlamydia (Eagle Minimum Essential Culture Medium containing 1 μg/ml cycloheximide) was added and cultivation was performed at 37° C. for 72 hours. After completion of cultivation, the culture medium was removed and the cells were fixed with methanol and then stained with FITC-labeled anti-C. trachomatis monoclonal antibody, followed by counting the number of inclusion bodies of C. trachomatis.

As a result, it was found that (−)-epicatechin gallate and (−)-epigallocatechin gallate completely inhibited the growth of C. trachomatis in concentrations of 0.8 mg/ml and 1.6 mg/ml, respectively.

### EXAMPLE 3

Anti-Chlamydia agents were prepared in the following formulations. The compositional analysis values of “Polyphenon E” used as tea polyphenol are as follows.

Composition of “Polyphenon E”

| | |
|---|---|
| (−)-Epigallocatechin | 12% |
| (−)-Epicatechin | 9% |
| (−)-Epigallocatechin gallate | 53% |
| (−)-Gallocatechin gallate | 6% |
| (−)-Epicatechin gallate | 4% |

| Prescription Example 1 | | |
|---|---|---|
| Zinc oxide | 200 | g |
| Liquid paraffin | 30 | g |
| White beeswax | 32.5 | g |
| Sorbitan sesquioleate | 13 | g |
| White vaseline | 604.5 | g |
| “Polyphenon E” | 120 | g |
| Total amount | 1,000 | g |
| Prescription Example 2 | | |
| White vaseline | 400 | g |
| Cetanol | 100 | g |
| White beeswax | 50 | g |
| Sorbitan sesquioleate | 50 | g |
| Lauromacrogo1 | 5 | g |
| Ethyl paraoxybenzoate | 1 | g |
| (or methyl paraoxybenzoate) | | |
| Butyl paraoxybenzoate | 1 | g |
| (or propyl paraoxybenzoate) | | |
| “Polyphenon E” | 120 | g |
| Purified water | 273 | g |
| Total amount | 1,000 | g |
| Prescription Example 3 | | |
| White vaseline | 250 | g |
| Stearyl alcohol | 200 | g |
| Propylene glycol | 120 | g |
| Polyoxyethylene-hydrated castor | 40 | g |
| oil 60 | | |
| Glycerin monostearate | 10 | g |
| Methyl paraoxybenzoate | 1 | g |
| Propyl paraoxybenzoate | 1 | g |
| “Polyphenon E” | 120 | g |
| Purified water | 258 | g |
| Total amount | 1,000 | g |
| Prescription Example 4 | | |
| Beeswax | 330 | g |
| Vegetable oil | 550 | g |
| “Polyphenon E” | 120 | g |
| Total amount | 1,000 | g |
| Prescription Example 5 | | |
| White beeswax | 50 | g |
| Sorbitan sesquioleate | 20 | g |
| White vaseline | 810 | g |
| “Polyphenon E” | 120 | g |
| Total amount | 1,000 | g |
| Prescription Example 6 | | |
| Macrogol (polyethylene glycol) | 440 | g |
| 4,000 | | |
| Macrogol (polyethylene glycol) | 440 | g |
| 400 | | |
| “Polyphenon E” | 120 | g |
| Total amount | 1,000 | g |
| Prescription Example 7 | | |
| Stearic acid | 200 | g |
| Potassium hydroxide | 13 | g |
| Glycerin | 100 | g |
| Methyl paraoxybenzoate | 1 | g |
| Propyl paraoxybenzoate | 1 | g |
| “Polyphenon E” | 120 | g |
| Purified water | 565 | g |
| Total amount | 1,000 | g |
| Prescription Example 8 | | |
| Stearic acid | 150 | g |
| Isopropyl palmitate | 20 | g |
| Lanolin | 10 | g |
| Sorbitol | 56 | g |
| Potassium hydroxide | 10 | g |
| Methyl paraoxybenzoate | 1 | g |
| Propyl paraoxybenzoate | 1 | g |
| “Polyphenon E” | 120 | g |
| Purified water | 632 | g |
| Total amount | 1,000 | g |
| Prescription Example 9 | | |
| Stearic acid | 180 | g |
| Liquid paraffin | 20 | g |
| Lanolin | 5 | g |
| Sorbitan sesquioleate | 20 | g |
| Potassium hydroxide | 8 | g |
| Sorbitol | 35 | g |
| Methyl paraoxybenzoate | 1 | g |
| Propyl paraoxybenzoate | 1 | g |
| “Polyphenon E” | 120 | g |
| Purified water | 610 | g |
| Total amount | 1,000 | g |
| Prescription Example 10 | | |
| Boric acid | 20 | mg |
| Methyl paraoxybenzoate | 0.26 | mg |
| Propyl paraoxybenzoate | 0.14 | mg |
| “Polyphenon E” | 250 | mg |
| Purified water total amount | 1,000 | ml |
| Prescription Example 11 | | |
| Sodium dihydrogen phosphate | 5.6 | mg |
| anhydride | | |
| Sodium hydrogen phosphate | 2.84 | mg |
| anhydride | | |
| Methyl paraoxybenzoate | 0.26 | mg |
| Propyl paraoxybenzoate | 0.14 | mg |
| “Polyphenon E” | 250 | mg |
| Purified water total amount | 1,000 | ml |
| Prescription Example 12 | | |
| Methyl paraoxybenzoate | 0.26 | mg |
| Propyl paraoxybenzoate | 0.14 | mg |
| “Polyphenon E” | 250 | mg |
| Purified water total amount | 1,000 | ml |

INDUSTRTAL APPLICABILITY According to the present invention, a preventive and therapeutic medicine which is effective on Chlamydia infectious diseases and highly safe is provided. This medicine has no side effect nor the risk of emergence of resistant strains unlike the case where antibiotics are administered.

### INDUSTRIAL APPLICABILITY

According to the present invention, a preventive and therapeutic medicine which is effective on Chlamydia infectious diseases and highly safe is provided. This medicine has no side effect nor the risk of emergence of resistant strains unlike the case where antibiotics are administered.

1. An anti-Chlamydia agent, which comprises containing tea polyphenol.

2. The anti-Chlamydia agent as claimed in claim 1, wherein the tea polyphenol is at least one selected from the group consisting of (+)-catechin, (−)-catechin, (+)-gallocatechin, (+)-epigallocatechin, (+)-gallocatechin gallate, (+)-epigallocatechin gallate, (−)-epicatechin, (−)-epicatechin gallate, (−)-catechin gallate, (−)-epigallocatechin, (−)-gallocatechin, (−) -epigallocatechin gallate, (−) -gallocatechin gallate, teaflavin monogallate A, teaflavin monogallate B, teaflavin digallate, and free teaflavin.

3. The anti-Chlamydia agent as claimed in claim 1, wherein Chlamydia is Chlamydia trachomatis.

4. A preventive or therapeutic method for a Chlamydia infectious disease, characterized by administering a composition containing tea polyphenol in an amount effective for the therapy of a Chlamydia infectious disease on an affected part of a patient.

5. The preventive or therapeutic method as claimed in claim 4, wherein the composition is in the form of cream, paste, gel, ointment, milky lotion, solution or suspension.

6. The preventive or therapeutic method as claimed in claim 4, wherein the tea polyphenol in the composition is in a concentration of 0.2 to 50 mg/ml when used in the form of liquid, milky lotion, or the like or 0.2 to 200 mg/g when used in the form of cream, paste, gel, ointment or the like.
